(19) United States
(12) Patent Application Publication
(10) Pub. No.: US 2019/0201296 A1
(43) Pub. Date: Jul. 4, 2019

(54) COATING COMPOSITIONS HAVING HYDROPHILIC AND HYDROPHOBIC PARTICLES FOR USE IN INTRAORAL SCANNING METHODS

(71) Applicant: 3M INNOVATIVE PROPERTIES COMPANY, St. Paul, MN (US)

(72) Inventors: Yizhong Wang, Woodbury, MN (US); Zhisheng Yun, Woodbury, MN (US); Eric S. Hansen, Arden Hills, MN (US); Joel D. Oxman, Minneapolis, MN (US); James D. Hansen, White Bear Lake, MN (US); Bradley D. Craig, Lake Elmo, MN (US); Tao Gong, Woodbury, MN (US)

(21) Appl. No.: 16/301,211

(22) PCT Filed: May 15, 2017
(86) PCT No.: PCT/US2017/032630

§ 371 Date: Nov. 13, 2018

Related U.S. Application Data
(60) Provisional application No. 62/342,747, filed on May 27, 2016.

(51) Int. Cl. A61K 6/00; A61K 6/10; A61C 9/00

(52) U.S. Cl. CPC A61K 6/002; A61C 9/0053; A61K 6/0017; A61K 6/10

(57) ABSTRACT

Disclosed herein are aqueous dental coating compositions and methods for enhancing contrast of intraoral surfaces for 3-dimensional digital scanning. The compositions include: an aqueous solvent system; a water soluble polymer present at no greater than 5 wt. %, based on the total weight of the aqueous dental coating composition; hydrophilic particles; and hydrophobic particles, wherein the aqueous dental coating composition is a dispersion or a dispersion of the hydrophilic particles and hydrophobic particles in the aqueous solvent system can be formed by shaking the composition for 30 seconds.

### BACKGROUND

Certain intraoral scanning systems rely upon a powder that is applied to the teeth before video imaging and subsequent three-dimensional (3D) digital impressions or models can be successfully generated. One of the challenges for successfully generating digital impressions using a multiple view geometry method is that a sufficient number of features with sufficient contrast must be obtained in the video images of the teeth. There is a wide range of teeth color and texture in the patient populace in conjunction with practical resolution limitations of the camera system that can require the application of a powder to homogenize a wide variety of imaging conditions.

Some of these scanning systems have used a white powder including titanium dioxide particles. The white powder has been deemed sufficient to provide the consistent scattering of light from the scanning wand and texture or granularity that would lead to adequate features in the video images. However, handling of particles in aerosols, for example can be messy and cause difficulty in handling, typically utilizing expensive and specially designed delivery devices to dust dental tissues. Such titanium dioxide powders can also lack sufficient adhesion to dental structures, and thus can be easily moved around by saliva, the tongue, and/or the scanner wand.

There is a continuing need for materials and methods for enhancing contrast of teeth for intraoral scanning.

### SUMMARY

In one aspect, the present disclosure provides an aqueous dental coating composition including: an aqueous solvent system; a water soluble polymer present at no greater than 5 wt. %, based on the total weight of the aqueous dental coating composition; hydrophilic particles; and hydrophobic particles, wherein the aqueous dental coating composition is a dispersion or a dispersion of the hydrophilic particles and hydrophobic particles in the aqueous solvent system can be formed by shaking the composition for 30 seconds.

In another aspect, the present disclosure provides a method of intraoral scanning including: applying an aqueous dental coating composition as described herein to an intraoral surface or model thereof to form a coating on the surface; and scanning the coated surface to form a three dimensional representation of the intraoral surface.

The aqueous dental coating compositions can be advantageous for ease of application, along with enhanced contrast enhancement, especially when used with, for example, a multiple view geometry method (e.g., multiple camera or stereo camera oral scanners) such as the oral scanner available under the trade designation 3M TRUE DEFINITION Scanner for digital impressioning from 3M. At least in some embodiments, the compositions and methods disclosed herein can overcome at least some of the problems encountered with application of powders (e.g., messy and difficult handling, utilization of expensive and specially designed delivery devices to dust dental tissues; and lack of sufficient adhesion to dental structures).

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

In this application, terms such as “a,” “an,” and “the” are not intended to refer to only a singular entity, but include the general class of which a specific example may be used for illustration. The terms “a,” “an,” and “the” are used interchangeably with the term “at least one.”

The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

Also herein, all numbers are assumed to be modified by the term “about” and in certain situations by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Also, as used herein in connection with a measured quantity, the term “approximately” refers to that variation in the measured quantity as would be expected by the skilled artisan making the measurement and exercising a level of care commensurate with the objective of the measurement and the precision of the measuring equipment used.

Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

As used herein, the term “room temperature” refers to a temperature of 20° C. to 25° C., or more often to a temperature of 21° C.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS

Disclosed herein are aqueous dental coating compositions and methods for enhancing contrast of intraoral surfaces for 3-dimensional digital scanning. As used herein, the term “intraoral surface” refers to a surface of a dental object in the oral cavity of a subject. The term “dental object”, as used herein, is intended to refer broadly to subject matter specific to dentistry. This may include intraoral structures such as dentition, and more typically human dentition, such as individual teeth, quadrants, full arches, pairs of arches which may be separate or in occlusion of various types, soft tissue, and the like, as well bones and any other supporting or surrounding structures. As used herein, the term “intraoral structures” refers to both natural structures within a mouth as described above and artificial structures such as any of the dental objects described below that might be present in the mouth. Dental objects may include “restorations,” which may be generally understood to include components that restore the structure or function of existing dentition, such as crowns, bridges, veneers, inlays, onlays, amalgams, composites, and various substructures such as copings and the like, as well as temporary restorations for use while a permanent restoration is being fabricated. Dental objects may also include a “prosthesis” that replaces dentition with removable or permanent structures, such as dentures, partial dentures, implants, retained dentures, and the like. Dental objects may also include “appliances” used to correct, align, or otherwise temporarily or permanently adjust dentition, such as removable orthodontic appliances, surgical stents, bruxism appliances, snore guards, indirect bracket placement appliances, and the like. Dental objects may also include “hardware” affixed to dentition for an extended period, such as implant fixtures, implant abutments, orthodontic brackets, and other orthodontic components. Dental objects may also include “interim components” of dental manufacture such as dental models (full and/or partial), wax-ups, investment molds, and the like, as well as trays, bases, dies, and other components employed in the fabrication of restorations, prostheses, and the like. Dental objects may also be categorized as natural dental objects such as the teeth, bone, and other intraoral structures described above or as artificial dental objects such as the restorations, prostheses, appliances, hardware, and interim components of dental manufacture as described above.

The aqueous dental coating compositions include: an aqueous solvent system; a water soluble polymer present at no greater than 5 wt. %, based on the total weight of the aqueous dental coating composition; hydrophilic particles; and hydrophobic particles, wherein the aqueous dental coating composition is a dispersion or a dispersion of the hydrophilic particles and hydrophobic particles in the aqueous solvent system can be formed by shaking the composition for 30 seconds.

The aqueous dental coating compositions include an aqueous solvent system. In some embodiments, the aqueous solvent system consists of water. In certain embodiments, the aqueous solvent system includes, for example, one or more cosolvents in addition to water. The use of cosolvents can be advantageous, for example, to enhance the drying properties of the coating after application to an intraoral surface. In certain embodiments, cosolvents can also enhance the dissolution of the water soluble polymer and/or enhance the dispersiblity of the particles.

A wide variety of water miscible cosolvents can be used. Particularly useful cosolvents include, for example, lower alkanols such as ethanol.

For exemplary embodiments in which the aqueous solvent system includes water and ethanol, the solvent system can include at least 1 wt. % ethanol, at least 10 wt. % ethanol, or at least 20 wt. % ethanol, based on the total weight of the water and ethanol. For exemplary embodiments in which the aqueous solvent system includes water and ethanol, the solvent system can include at most 35 wt. % ethanol, at most 30 wt. % ethanol, or at most 25 wt. % ethanol, based on the total weight of the water and ethanol. Higher amounts of ethanol as a cosolvent are typically avoided to minimize any burning or discomfort to the patient during application or rinsing procedures.

The aqueous dental coating compositions also include a water soluble polymer. In some embodiments, the water soluble polymer advantageously enhances the bonding of the particles to the intraoral surface after application and drying of the aqueous dental coating composition to the intraoral surface.

A wide variety of safe and effective water soluble polymers can be used in the aqueous dental coating compositions. Particularly useful water soluble polymers include, but are not limited to, modified cellulose, polyethylene glycol (PEG), polyacrylic acid, polyvinyl alcohol (PVA), polyethylene oxide-polypropylene oxide copolymers (PEO-PPO), polyethylene oxide-vinyl acetate copolymers (PEO-PVAc), polyvinyl pyrrolidinone (PVP), Eudragit acrylates, or combinations thereof.

The aqueous dental coating composition includes no greater than 5 wt. % of a water soluble polymer, based on the total weight of the aqueous dental coating composition. In certain embodiments, the aqueous dental coating composition includes at least 0.1 wt. % water soluble polymer, at least 0.5 wt. % water soluble polymer, or at least 1.5 wt. % water soluble polymer, based on the total weight of the aqueous dental coating composition. In certain embodiments, the aqueous dental coating composition includes at most 5 wt. % water soluble polymer, at most 3 wt. % water soluble polymer, or at most 2 wt. % water soluble polymer, based on the total weight of the aqueous dental coating composition.

The aqueous dental coating composition further includes hydrophilic particles and hydrophobic particles. The combination of both hydrophilic particles and hydrophobic particles can be advantageous for coating intraoral surfaces having enhanced contrast for 3-dimensional digital scanning. In some embodiments, the hydrophilic particles and the hydrophobic particles are substantially non-absorptive in the visible range. As used herein, “substantially non-absorptive” in the visible range means that the particles absorb less than 50% of incident light having a wavelength of 400 nm to 700 nm. In some embodiments, the hydrophilic particles and the hydrophobic particles are tooth-colored. As used herein, “tooth-colored” refers to the color of a normal, vital, healthy tooth.

As used herein, “hydrophilic” particles include particles that can be readily dispersed in water. In certain embodiments, hydrophilic particles can be readily dispersed in water using Test Method A described herein.

As used herein, “hydrophobic” particles include particles that cannot be readily dispersed in water. In certain embodiments, hydrophobic particles cannot be readily dispersed in water using Test Method A described herein.

A wide variety of hydrophilic particles can be used in the aqueous dental coating compositions described herein. Useful hydrophilic particles include organic particles, inorganic particles, and combinations thereof. Hydrophilic particles can be discrete primary particles, agglomerated particles, or aggregated particles. In certain embodiments, hydrophilic particles can be core-shell particles.

Exemplary organic hydrophilic particles include polymeric particles that can be in the form of beads, spheres, rods, or any other form desired. The polymeric particles can be unfilled, or filled with other fillers such as inorganic fillers to form composite particles.

Exemplary inorganic hydrophilic particles include, but are not limited to, metal particles, metal oxide particles, metal hydroxide particles, metal nitride particles, metal sulfide particles, metal phosphide particles, and metal salts of organic and/or inorganic acids.

Hydrophilic particles can be surface modified particles or non-surface modified particles. Surface modified hydrophilic particles include particles that have been coated with another material or otherwise treated (e.g., by oxidation or other reactions of surface groups) to provide particles in which the surface properties have been modified. In certain embodiments, a particle can be surface treated to provide hydrophilic properties. A wide variety of surface treatments can be used for hydrophilic particles. Exemplary hydrophilic surface treatments include, but are not limited to, alcohols and polyols, glycols (e.g., ethylene glycol), polyethylene glycols, modified polyethylene glycols, amino acids, silane modified amino acids, and water soluble polymers as described herein. Particularly useful hydrophilic particles include, for example, non-surface treated TiO₂ particles.

In some embodiments, the hydrophilic particles have an average size of 0.01 to 10 microns. In some embodiments, the hydrophilic particles have an average size of at least 0.01 micron, at least 0.05 micron, or at least 0.5 micron. In some embodiments, the hydrophilic particles have an average size of at most 10 microns, at most 5 microns, or at most 3 microns.

In some embodiments, the aqueous dental coating composition includes 5 to 25 wt. % of the hydrophilic particles, based on the total weight of the aqueous dental coating composition. In certain embodiments, the aqueous dental coating composition includes at least 5 wt. % of the hydrophilic particles, at least 10 wt. % of the hydrophilic particles, or at least 15 wt. % of the hydrophilic particles, based on the total weight of the aqueous dental coating composition. In certain embodiments, the aqueous dental coating composition includes at most 25 wt. % of the hydrophilic particles, at most 20 wt. % of the hydrophilic particles, or at most 18 wt. % of the hydrophilic particles, based on the total weight of the aqueous dental coating composition.

A wide variety of hydrophobic particles can be used in the aqueous dental coating compositions described herein. Useful hydrophobic particles include organic particles, inorganic particles, and combinations thereof. Hydrophobic particles can be discrete primary particles, agglomerated particles, or aggregated particles. In certain embodiments, hydrophobic particles can be core-shell particles.

Exemplary organic hydrophobic particles include polymeric particles that can be in the form of beads, spheres, rods, or any other form desired. Hydrophobic polymeric particles can be unfilled, or filled with other fillers such as inorganic fillers to form composite particles.

Exemplary inorganic hydrophobic particles include, but are not limited to, metal particles, metal oxide particles, metal hydroxide particles, metal nitride particles, metal sulfide particles, metal phosphide particles, and metal salts of organic and/or inorganic acids.

Hydrophobic particles can be surface modified particles or non-surface modified particles. Surface modified hydrophobic particles include particles that have been coated with another material or otherwise treated (e.g., by oxidation or other reactions of surface groups) to provide particles in which the surface properties have been modified. In certain embodiments, a particle can be surface treated to provide hydrophobic properties. A wide variety of surface treatments can be used for hydrophobic particles. Exemplary hydrophobic surface treatments include, but are not limited to, alkyl or aryl carboxylates (e.g., decanoate salts, dodecanoate salts, and benzoate salts), alkyl sulfates (e.g., lauryl sulfate salts), alkyl or aryl sulfonates (e.g., lauryl sulfonate salts and benzene sulfonate salts), silanes (e.g., alkyl trialkoxy silanes), and siloxanes. Particularly useful hydrophobic particles include surface treated tricalcium phosphate (TCP) particles (e.g., surface treated with sodium lauryl sulfate and/or sodium lauryl sulfonate).

In some embodiments, the hydrophobic particles have an average size of 0.01 to 10 microns. In some embodiments, the hydrophobic particles have an average size of at least 0.01 micron, at least 0.05 micron, or at least 0.5 micron. In some embodiments, the hydrophobic particles have an average size of at most 10 microns, at most 5 microns, or at most 3 microns.

In some embodiments, the aqueous dental coating composition includes 1 to 15 wt. % of the hydrophobic particles, based on the total weight of the aqueous dental coating composition. In certain embodiments, the aqueous dental coating composition includes at least 1 wt. % of the hydrophobic particles, at least 3 wt. % of the hydrophobic particles, or at least 5 wt. % of the hydrophobic particles, based on the total weight of the aqueous dental coating composition. In certain embodiments, the aqueous dental coating composition includes at most 15 wt. % of the hydrophobic particles, at most 10 wt. % of the hydrophobic particles, or at most 5 wt. % of the hydrophobic particles, based on the total weight of the aqueous dental coating composition.

The aqueous dental coating compositions described herein may optionally include various additives known in the art including, but not limited to, flavorants, fluoridating agents, buffering agents, numbing agents, remineralization agents, desensitization agents, colorants, or combinations thereof. The presence of a colorant can aid in detecting that the aqueous composition has coated all the desired intraoral surfaces. The intensity of a colorant can also aid in detecting the uniformity of the coating on the intraoral surfaces.

In some embodiments, particles can be dispersed in the aqueous dental coating composition by a variety of methods known in the art. Useful methods can include, but are not limited to, agitation, shaking, ball milling, ultrasonication of the various components, or combinations thereof, typically at room temperature for a time effective to prepare the dispersion.

The viscosity of the aqueous dental coating compositions is typically low to enable application of a thin layer of the aqueous coating composition on the intraoral surface. In some embodiments, after the dispersion has been formed, a portion or all of the fillers may settle out during storage. Particularly useful aqueous dental coating compositions include those in which the particles can be redispersed by manually shaking the composition for 30 seconds.

The present disclosure further provides a method of intraoral scanning including: applying an aqueous dental coating composition as described herein to an intraoral surface or model thereof to form a coating on the surface; and scanning the coated surface to form a three dimensional representation of the intraoral surface.

A wide variety of methods can be employed to apply the aqueous dental coating composition on the intraoral surface. Exemplary methods include brushing, rinsing, sponging, spraying, or a combination thereof. In some embodiments, the aqueous dental coating composition can be applied from the composition's container or dispenser, such as a bottle, syringe, or tube. In some embodiments, a mini dental brush, a dental brush, microfiber, foam or sponge applicator, a specially designed notched brush to fit the tooth shape for fast swabbing across the arch, or a cotton Q tip can be used to rub the surface of the intraoral surface and leave a thin layer of coating on the surface. In some other embodiments, a tray applicator, a dental tray, a sponge tray, or a dental strip filled with the aqueous dental coating composition can be used. The aqueous dental coating composition can cover the intraoral surface and leave a layer of coating on the surface. In other embodiments, the aqueous dental coating composition can be sprayed (e.g. air-brushing) with a spray device or aerosol applicator onto the intraoral surface. In other embodiments, the aqueous dental coating composition can be directly painted onto the intraoral surface with a brush tip attached to a syringe. In yet other embodiments, the aqueous dental coating composition can be applied as a rinse. Once applied to the intraoral surface, the aqueous dental coating composition can be allowed to dry, or dried by blowing air, typically within 30 seconds.

In some embodiments, the aqueous dental coating composition is capable of forming a film on an intraoral surface after application to the intraoral surface and drying. In some embodiments, the aqueous dental coating composition is capable of forming a discontinuous film on an intraoral surface after application to the intraoral surface and drying. In some other embodiments, the aqueous dental coating composition is capable of forming the film in less than about 30 seconds after application, when dried with a stream of compressed air. In some embodiments, the dried coating has an average thickness of at most 10 microns.

The aqueous dental coating composition are particularly useful with digital scanners utilizing multiple view geometry methods (e.g., multiple cameras or stereo camera scanners). Exemplary scanning systems are described in U.S. Pat. No. 7,698,014 B2 (Dunne et al.), U.S. Pat. No. 7,813,591 B2 (Paley et al.), U.S. Pat. No. 7,840,042 B2 (Kriveshko et al.), U.S. Pat. No. 7,912,257 B2 (Paley et al.), U.S. Pat. No. 7,940,260 B2 (Kriveshko), U.S. Pat. No. 7,956,862 B2 (Zhang et al.), U.S. Pat. No. 8,454,365 B2 (Boerjes et al.), U.S. Pat. No. 8,503,763 B2 (Zhang et al.), U.S. Pat. No. 8,803,958 B2 (Zhang et al.), U.S. Pat. No. 8,830,309 B2 (Rohaly et al.), U.S. Pat. No. 8,866,883 B2 (Rohaly et al.), and U.S. Pat. No. 9,191,648 B2 (Kriveshko et al.); and U.S. Patent Application Pub. Nos. 2004/0155975 A1 (Hart et al.), 2010/0281370 A1 (Rohaly et al.), and 2011/0043613 A1 (Rohaly et al.). An exemplary oral scanner is available under the trade designation 3M TRUE DEFINITION Scanner for digital impressioning from 3M.

After scanning, in certain embodiments the applied coating can be readily removed from the intraoral surface by rinsing and/or brushing with water.

### ILLUSTRATIVE EMBODIMENTS OF THE PRESENT DISCLOSURE

Various embodiments are disclosed that can provide aqueous dental coating compositions and method of using same.

Embodiment 1A is an aqueous dental coating composition comprising: an aqueous solvent system; a water soluble polymer present at no greater than 5 wt. %, based on the total weight of the aqueous dental coating composition; hydrophilic particles; and hydrophobic particles, wherein the aqueous dental coating composition is a dispersion or a dispersion of the hydrophilic particles and hydrophobic particles in the aqueous solvent system can be formed by shaking the composition for 30 seconds.

Embodiment 2A is the aqueous dental coating composition of embodiment 1A, wherein the hydrophilic particles and the hydrophobic particles are substantially non-absorptive in the visible range.

Embodiment 3A is the aqueous dental coating composition of embodiment 1A or 2A, wherein the hydrophilic particles and the hydrophobic particles absorb less than 50% of incident light having a wavelength of 400 nm to 700 nm.

Embodiment 4A is the aqueous dental coating composition of any one of embodiments 1A to 3A, wherein the hydrophilic particles and the hydrophobic particles are tooth-colored.

Embodiment 5A is the aqueous dental coating composition of any one of embodiments 1A to 4A, wherein the aqueous solvent system comprises water, or water and ethanol.

Embodiment 6A is the aqueous dental coating composition of any one of embodiments 1A to 5A, wherein the aqueous solvent system comprises 0 to 35 wt. % ethanol, based on the total weight of water and ethanol.

Embodiment 7A is the aqueous dental coating composition of any one of embodiments 1A to 6A, wherein the solvent system includes at least 1 wt. % ethanol, based on the total weight of the water and ethanol.

Embodiment 8A is the aqueous dental coating composition of any one of embodiments 1A to 7A, wherein the solvent system includes at least 10 wt. % ethanol, based on the total weight of the water and ethanol.

Embodiment 9A is the aqueous dental coating composition of any one of embodiments 1A to 8A, wherein the solvent system includes at least 20 wt. % ethanol, based on the total weight of the water and ethanol.

Embodiment 10A is the aqueous dental coating composition of any one of embodiments 1A to 9A, wherein the solvent system includes at most 35 wt. % ethanol, based on the total weight of the water and ethanol.

Embodiment 11A is the aqueous dental coating composition of any one of embodiments 1A to 10A, wherein the solvent system includes at most 30 wt. % ethanol, based on the total weight of the water and ethanol.

Embodiment 12A is the aqueous dental coating composition of any one of embodiments 1A to 11A, wherein the solvent system includes at most 25 wt. % ethanol, based on the total weight of the water and ethanol.

Embodiment 13A is the aqueous dental coating composition of any one of embodiments 1A to 12A, wherein the aqueous dental coating composition comprises 0.1 to 5 wt. % of the water soluble polymer, based on the total weight of the aqueous dental coating composition.

Embodiment 14A is the aqueous dental coating composition of any one of embodiments 1A to 13A, wherein the aqueous dental coating composition includes at least 0.1 wt. % water soluble polymer, based on the total weight of the aqueous dental coating composition.

Embodiment 15A is the aqueous dental coating composition of any one of embodiments 1A to 14A, wherein the aqueous dental coating composition includes at least 0.5 wt. % water soluble polymer, based on the total weight of the aqueous dental coating composition.

Embodiment 16A is the aqueous dental coating composition of any one of embodiments 1A to 15A, wherein the aqueous dental coating composition includes at least 1.5 wt. % water soluble polymer, based on the total weight of the aqueous dental coating composition.

Embodiment 17A is the aqueous dental coating composition of any one of embodiments 1A to 16A, wherein the aqueous dental coating composition includes at most 5 wt. % water soluble polymer, based on the total weight of the aqueous dental coating composition.

Embodiment 18A is the aqueous dental coating composition of any one of embodiments 1A to 17A, wherein the aqueous dental coating composition includes at most 3 wt. % water soluble polymer, based on the total weight of the aqueous dental coating composition.

Embodiment 19A is the aqueous dental coating composition of any one of embodiments 1A to 18A, wherein the aqueous dental coating composition includes at most 2 wt. % water soluble polymer, based on the total weight of the aqueous dental coating composition.

Embodiment 20A is the aqueous dental coating composition of any one of embodiments 1A to 19A, wherein the water soluble polymer is selected from the group consisting of modified cellulose, polyethylene glycol (PEG), polyacrylic acid, polyvinyl alcohol (PVA), polyethylene oxide-polypropylene oxide copolymers (PEO-PPO), polyethylene oxide-vinyl acetate copolymers (PEO-PVAc), polyvinyl pyrrolidinone (PVP), and combinations thereof.

Embodiment 21A is the aqueous dental coating composition of any one of embodiments 1A to 20A, wherein the hydrophilic particles have an average size of 0.01 to 10 microns.

Embodiment 22A is the aqueous dental coating composition of any one of embodiments 1A to 21A, wherein the hydrophilic particles have an average size of at least 0.01 micron.

Embodiment 23A is the aqueous dental coating composition of any one of embodiments 1A to 22A, wherein the hydrophilic particles have an average size of at least 0.05 micron.

Embodiment 24A is the aqueous dental coating composition of any one of embodiments 1A to 23A, wherein the hydrophilic particles have an average size of at least 0.5 micron.

Embodiment 25A is the aqueous dental coating composition of any one of embodiments 1A to 24A, wherein the hydrophilic particles have an average size of at most 10 microns.

Embodiment 26A is the aqueous dental coating composition of any one of embodiments 1A to 25A, wherein the hydrophilic particles have an average size of at most 5 microns.

Embodiment 27A is the aqueous dental coating composition of any one of embodiments 1A to 26A, wherein the hydrophilic particles have an average size of at most 3 microns.

Embodiment 28A is the aqueous dental coating composition of any one of embodiments 1A to 27A, wherein the hydrophilic particles comprise inorganic particles.

Embodiment 29A is the aqueous dental coating composition of any one of embodiments 1A to 28A, wherein the hydrophilic particles comprise TiO₂ particles.

Embodiment 30A is the aqueous dental coating composition of any one of embodiments 1A to 29A, wherein the aqueous dental coating composition comprises 5 to 25 wt. % of the hydrophilic particles, based on the total weight of the aqueous dental coating composition.

Embodiment 31A is the aqueous dental coating composition of any one of embodiments 1A to 30A, wherein the aqueous dental coating composition includes at least 5 wt. % of the hydrophilic particles, based on the total weight of the aqueous dental coating composition.

Embodiment 32A is the aqueous dental coating composition of any one of embodiments 1A to 31A, wherein the aqueous dental coating composition includes at least 10 wt. % of the hydrophilic particles, based on the total weight of the aqueous dental coating composition.

Embodiment 33A is the aqueous dental coating composition of any one of embodiments 1A to 32A, wherein the aqueous dental coating composition includes at least 15 wt. % of the hydrophilic particles, based on the total weight of the aqueous dental coating composition.

Embodiment 34A is the aqueous dental coating composition of any one of embodiments 1A to 33A, wherein the aqueous dental coating composition includes at most 25 wt. % of the hydrophilic particles, based on the total weight of the aqueous dental coating composition.

Embodiment 35A is the aqueous dental coating composition of any one of embodiments 1A to 34A, wherein the aqueous dental coating composition includes at most 20 wt. % of the hydrophilic particles, based on the total weight of the aqueous dental coating composition.

Embodiment 36A is the aqueous dental coating composition of any one of embodiments 1A to 35A, wherein the aqueous dental coating composition includes at most 18 wt. % of the hydrophilic particles, based on the total weight of the aqueous dental coating composition.

Embodiment 37A is the aqueous dental coating composition of any one of embodiments 1A to 36A, wherein the hydrophobic particles have an average size of 0.01 to 10 microns.

Embodiment 38A is the aqueous dental coating composition of any one of embodiments 1A to 37A, wherein the hydrophobic particles have an average size of at least 0.01 micron.

Embodiment 39A is the aqueous dental coating composition of any one of embodiments 1A to 38A, wherein the hydrophobic particles have an average size of at least 0.05 micron.

Embodiment 40A is the aqueous dental coating composition of any one of embodiments 1A to 39A, wherein the hydrophobic particles have an average size of at least 0.5 micron.

Embodiment 41A is the aqueous dental coating composition of any one of embodiments 1A to 40A, wherein the hydrophobic particles have an average size of at most 10 microns.

Embodiment 42A is the aqueous dental coating composition of any one of embodiments 1A to 41A, wherein the hydrophobic particles have an average size of at most 5 microns.

Embodiment 43A is the aqueous dental coating composition of any one of embodiments 1A to 42A, wherein the hydrophobic particles have an average size of at most 3 microns.

Embodiment 44A is the aqueous dental coating composition of any one of embodiments 1A to 43A, wherein the hydrophobic particles comprise inorganic particles.

Embodiment 45A is the aqueous dental coating composition of any one of embodiments 1A to 44A, wherein the hydrophobic particles comprise surface treated inorganic particles.

Embodiment 46A is the aqueous dental coating composition of any one of embodiments 1A to 45A, wherein the hydrophobic particles comprise tricalcium phosphate particles surface treated with sodium lauryl sulfate.

Embodiment 47A is the aqueous dental coating composition of any one of embodiments 1A to 46A, wherein the aqueous dental coating composition comprises 1 to 15 wt. % of the hydrophobic particles, based on the total weight of the aqueous dental coating composition.

Embodiment 48A is the aqueous dental coating composition of any one of embodiments 1A to 47A, wherein the aqueous dental coating composition includes at least 1 wt. % of the hydrophobic particles, based on the total weight of the aqueous dental coating composition.

Embodiment 49A is the aqueous dental coating composition of any one of embodiments 1A to 48A, wherein the aqueous dental coating composition includes at least 3 wt. % of the hydrophobic particles, based on the total weight of the aqueous dental coating composition.

Embodiment 50A is the aqueous dental coating composition of any one of embodiments 1A to 49A, wherein the aqueous dental coating composition includes at least 5 wt. % of the hydrophobic particles, based on the total weight of the aqueous dental coating composition.

Embodiment 51A is the aqueous dental coating composition of any one of embodiments 1A to 50A, wherein the aqueous dental coating composition includes at most 15 wt. % of the hydrophobic particles, based on the total weight of the aqueous dental coating composition.

Embodiment 52A is the aqueous dental coating composition of any one of embodiments 1A to 51A, wherein the aqueous dental coating composition includes at most 10 wt. % of the hydrophobic particles, based on the total weight of the aqueous dental coating composition.

Embodiment 53A is the aqueous dental coating composition of any one of embodiments 1A to 52A, wherein the aqueous dental coating composition includes at most 5 wt. % of the hydrophobic particles, based on the total weight of the aqueous dental coating composition.

Embodiment 1B is a method of intraoral scanning comprising: applying an aqueous dental coating composition according to any one of embodiments 1A to 53A to an intraoral surface or model thereof to form a coating on the surface; and scanning the coated surface to form a three dimensional representation of the intraoral surface.

Embodiment 2B is the method of embodiment 1B further comprising drying the applied aqueous dental coating composition to form the coating.

Embodiment 3B is the method of embodiment 2B, wherein the dried coating has an average thickness of at most 10 microns.

Embodiment 4B is the method of any one of embodiments 1B to 3B, wherein applying comprises brushing, rinsing, sponging, spraying, or a combination thereof.

Objects and advantages of this disclosure are further illustrated by the following non-limiting examples, but the particular materials and amounts thereof recited in these examples, as well as other conditions and details, should not be construed to unduly limit this disclosure.

### EXAMPLES

TABLE 1
List of materials

| COMPONENT | SOURCE | ADDRESS |
|---|---|---|
| Ethanol 190 proof | Columbus chemical | Ashville, OH |
| USP | industries Inc. | |
| Kollicoat IR polymer | BASF | Florham Park, NJ |
| TiO₂ 3328 BC | Brenntag specialties | South Plainfield, NJ |
| Micro TiO₂ | BRANDT | Bensenville, IL |
| | TECHNOLOGIES LLC | |
| Sodium fluoride USP | Phibro corporation | Teaneck, NJ |
| Fruit flavor | The Foote & Jenks | Camden, NJ |
| | corporation | |
| Sucralose | Sigma Aldrich | St Louis MO |

### Preparation and Characterization of Materials

### Sodium Lauryl Sulfate Surface Treated Tricalcium Phosphate (TCP-sls)

Sodium lauryl sulfate surface treated tricalcium phosphate (TCP-sls) with a TCP/SLS ratio of about 62:1.3 was prepared in a manner similar to that described in U.S. Pat. No. 9,023,373 B2 (Karlinsey). Briefly, ten 20 mm zirconia milling media balls, 250 gram of beta tricalcium phosphate, 4.4 g gram of sodium lauryl sulfate, and 200 ml of pentane were sealed in a milling jar. The jar was fixed in a milling station and milled at 375 revolutions per minute for 2 hours. The milling media balls were removed, and the remaining liquid was then dried using a vacuum oven to remove the solvent.

### TiO₂ 3328 BC

TiO₂ 3328 BC is a purified grade of titanium dioxide with anatase crystal structure from Brenntag specialties. It has average particles size of 0.3 micron and a maximum particle size 1.0 micron, with a specific gravity of 3.90.

### Micro TiO₂

Micro TiO₂ is from Brandt Technologies LLC. It is a mixture of TiO₂ (max 85%), Alumina (6-9%), and Zirconia (1.2-2.2%), TiO₂ has oil absorption of 35-45 gram/100 gram TiO₂. Titanium Dioxide Micro MT 500 H.D. Average primary particle size is 30 nanometers.

### Kollicoat IR Polymer

Kollicoat IR polymer is a polyethylene glycol (PEG) grafted with polyvinyl alcohol (PVA) polymer.

### Preparation of Polymer Solutions

Polymer solutions were prepared by charging all chemicals in a glass bottle, and then mixing by magnetic stirring for 24 hours at room temperature to form a clear solution.

TABLE 2
Polymer solution (PS) preparation (gram)

| | | | Control | |
|---|---|---|---|---|
| | | | (no | |
| COMPONENTS | PS-1 | PS-2 | polymer) | PS-3 |
| 190 proof | 90 | 0 | 0 | 0 |
| ethanol | | | | |
| Kollicoat IR | 10 | 2.5 | 0 | 7.5 |
| polymer | | | | |
| DI water | 400 | 497.5 | 490 | 492.5 |

### EXAMPLES

### Dispersion Stability Testing

The dispersion stability was tested by shaking the particles and polymer solutions to determine whether they could be redispersed in the aqueous media after sitting for 24 hours. If the particles could be redispersed in the aqueous media, the composition had good dispersion capability. If the particles formed a hard sediment at the bottom, the composition had a bad dispersion capability.

### Scanability Testing

The scanability was tested using a 3M TRUE DEFINITION oral scanner to evaluate whether the scanner could easily capture the image of a typodont painted with the dispersion. The scanability was ranked high, medium, or low. High scanability means that the oral scanner captured the 3D images smoothly and quickly when the wand moved across the coated typodont surface, which is comparable to other state of the art scanning methods. Medium scanability means that the wand of the scanner needed to be moved slowly and repeatedly to capture the 3D images. Low scanability means that the wand of the scanner had difficulty capturing the typodont image, resulting in the inability to create a smooth 3D typodont profile.

To prepare the typodont for the scanability study, a brush was dipped into the dispersion and used to thoroughly paint the surface of the typodont.

TABLE 3
Particle dispersions and their properties (gram)

| Components | EX-1 | EX-2 | CE-1 | CE-2 | CE-3 | CE-4 | CE-5 |
|---|---|---|---|---|---|---|---|
| PS-1 | 0 | 0 | 0 | 0 | 40 | 40 | 0 |
| PS-2 | 0 | 0 | 40 | 0 | 0 | 0 | 0 |
| PS-3 | 80 | 120 | 0 | 0 | 0 | 0 | 40 |
| Control | 0 | 0 | 0 | 40 | 0 | 0 | 0 |
| Micro TiO₂ | 0 | 0 | 0 | 0 | 5 | 10 | 0 |
| TiO₂ 3328 | 7.5 | 14 | 8.3 | 10 | 0 | 0 | 0 |
| TCP-sls | 1.25 | 2.0 | 0 | 0 | 0 | 0 | 8.3 |
| Sucralose | 0 | 0.1 | 0 | 0 | 0 | 0 | 0 |
| Sodium | 0 | 0.024 | 0 | 0 | 0 | 0 | 0 |
| fluoride | | | | | | | |
| Fruit flavor | 0 | 2.0 | 0 | 0 | 0 | 0 | 0 |
| Dispersion | Good | Good | Good | Good | Good | Good | Bad |
| capability | | | | | | | |
| Scanability | High | High | Low | Low | Medium | Medium | N/A |

### Hydrophilicity and Dispersibility Testing

### Test Method A

To determine the dispersibility of a particle in water, 1 gram of particles was added to 9 grams of water in a glass vial and shaken for 10 seconds. The vials were then allowed to sit for 24 hours, then shaken again for 30 seconds to visually determine whether the components could be redispersed in water again.

TCP and TiO₂ without surface treatment were easily dispersed to form a white dispersion. TCP-sls was difficult to redisperse in water after forming a layer of sediment at the bottom of the vials. Compared with TCP and TCP-sls, TCP without hydrophobic surface treatment could easily form a dispersion in water by a simple shaking of the samples. However, TCP-sls with hydrophobic surface treatment could not be easily dispersed in water by a simple shaking of the sample.

TABLE 4
Compositions for Hydrophilicity and Dispersiblity Testing (grams)

| COMPONENT | EX-3 | EX-4 | CE-6 |
|---|---|---|---|
| DI water | 9 | 9 | 9 |
| TCP | 1 | 0 | 0 |
| TCP-sls | 0 | 0 | 1 |
| TiO₂ 3328 | 0 | 1 | 0 |
| dispersion | Yes | Yes | No |

All cited references, patents, or patent applications in the above application for letters patent are herein incorporated by reference in their entirety in a consistent manner. In the event of inconsistencies or contradictions between portions of the incorporated references and this application, the information in the preceding description shall control. The preceding description, given in order to enable one of ordinary skill in the art to practice the claimed disclosure, is not to be construed as limiting the scope of the disclosure, which is defined by the claims and all equivalents thereto.

1. An aqueous dental coating composition comprising:
an aqueous solvent system;
a water soluble polymer present at no greater than 5 wt. %, based on the total weight of the aqueous dental coating composition;
hydrophilic particles; and
hydrophobic particles,
wherein the aqueous dental coating composition is a dispersion or a dispersion of the hydrophilic particles and hydrophobic particles in the aqueous solvent system can be formed by shaking the composition for 30 seconds.

2. The aqueous dental coating composition of claim 1, wherein the hydrophilic particles and the hydrophobic particles are substantially non-absorptive in the visible range.

3. The aqueous dental coating composition of claim 1, wherein the hydrophilic particles and the hydrophobic particles are tooth-colored.

4. The aqueous dental coating composition of claim 1, wherein the aqueous solvent system comprises water, or water and ethanol.

5. The aqueous dental coating composition of claim 4, wherein the aqueous solvent system comprises 0 to 35 wt. % ethanol, based on the total weight of water and ethanol.

6. The aqueous dental coating composition of claim 1, wherein the aqueous dental coating composition comprises 0.1 to 5 wt. % of the water soluble polymer, based on the total weight of the aqueous dental coating composition.

7. The aqueous dental coating composition of claim 1, wherein the water soluble polymer is selected from the group consisting of modified cellulose, polyethylene glycol (PEG), polyacrylic acid, polyvinyl alcohol (PVA), polyethylene oxide-polypropylene oxide copolymers (PEO-PPO), polyethylene oxide-vinyl acetate copolymers (PEO-PVAc), polyvinyl pyrrolidinone (PVP), and combinations thereof.

8. The aqueous dental coating composition of claim 1, wherein the hydrophilic particles have an average size of 0.01 to 10 microns.

9. The aqueous dental coating composition of claim 1, wherein the hydrophilic particles comprise inorganic particles.

10. The aqueous dental coating composition of claim 1, wherein the hydrophilic particles comprise TiO₂ particles.

11. The aqueous dental coating composition of claim 1, wherein the aqueous dental coating composition comprises 5 to 25 wt. % of the hydrophilic particles, based on the total weight of the aqueous dental coating composition.

12. The aqueous dental coating composition of claim 1, wherein the hydrophobic particles have an average size of 0.01 to 10 microns.

13. The aqueous dental coating composition of claim 1, wherein the hydrophobic particles comprise inorganic particles.

14. The aqueous dental coating composition of claim 1, wherein the hydrophobic particles comprise surface treated inorganic particles.

15. The aqueous dental coating composition of claim 1, wherein the hydrophobic particles comprise tricalcium phosphate particles surface treated with sodium lauryl sulfate.

16. The aqueous dental coating composition of claim 1, wherein the aqueous dental coating composition comprises 1 to 15 wt. % of the hydrophobic particles, based on the total weight of the aqueous dental coating composition.

17. A method of intraoral scanning comprising:
applying an aqueous dental coating composition of claim 1, to an intraoral surface or model thereof to form a coating on the surface; and
scanning the coated surface to form a three dimensional representation of the intraoral surface.

18. The method of claim 17 further comprising drying the applied aqueous dental coating composition to form the coating.

19. The method of claim 18, wherein the dried coating has an average thickness of at most 10 microns.

20. The method of claim 17, wherein applying comprises brushing, rinsing, sponging, spraying, or a combination thereof.
